(19) United States
(12) Patent Application Publication
(10) Pub. No.: US 2021/0199237 A1
(43) Pub. Date: Jul. 1, 2021

(54) BASE FRAME AND POSITIONING HEAD FRAME

(71) Applicants: OUR UNITED CORPORATION, Shaanxi (CN); SMD Medical Co., Ltd., Hubei Province (CN)

(72) Inventors: Hui LIU, Shaanxi (CN); Xuemin HOU, Shaanxi (CN)

(21) Appl. No.: 17/130,100

(22) Filed: Dec. 22, 2020

(30) Foreign Application Priority Data
Dec. 27, 2019 (CN) 201922424863.2

(51) Int. Cl. F16M 11/22; F16M 11/20

(52) U.S. Cl. CPC F16M 11/22; F16M 11/2035; F16M 2200/08; F16M 2200/068; F16M 2200/027

(57) ABSTRACT

A base frame includes a base frame body and a connector. The base frame body has an opening therein. The connector is disposed at the opening of the base frame body to seal the opening of the base frame body, and the connector is made of an insulating material.

### CROSS-REFERENCE TO RELATED APPLICATION

This application claims priority to Chinese Patent Application No. 201922424863.2, field on Dec. 27, 2019, which is incorporated herein by reference in its entirety.

### TECHNICAL FIELD

The present disclosure relates to the field of medical instruments, and in particular, to a base frame and a positioning head frame.

### BACKGROUND

In modern society, with the rapid development of computers and imaging technologies, radiotherapy technology has continued to mature, and treatment accuracy of the radiotherapy technology has become higher and higher. The radiotherapy technology includes three-dimensional conformal radiation therapy (3DCRT) technology, intensity-modulated radiation therapy (IMRT) technology, and image guided radiation therapy (IGRT) technology, etc.

### SUMMARY

In a first aspect, a base frame is provided. The base frame includes a base frame body and a connector. The base frame body has an opening therein, and the base frame body is made of a metal material. The connector is disposed at the opening of the base frame body to seal the opening of the base frame body, and the connector is made of an insulating material.

In a second aspect, a positioning head frame is provided. The positioning head frame includes a plurality of support members, a plurality of pins, and the base frame provided in the first aspect. The plurality of support members are fixedly connected to the base frame, and each pin is fixedly connected to one end of a corresponding support member.

### BRIEF DESCRIPTION OF THE DRAWINGS

In order to describe technical solutions of the present disclosure more clearly, the accompanying drawings to be used in some embodiments of the present disclosure will be introduced briefly. However, the accompanying drawings to be described below are merely accompanying drawings of some embodiments of the present disclosure, and a person of ordinary skill in the art can obtain other drawings according to those drawings. In addition, accompanying drawings in the following description may be regarded as schematic diagrams, and are not limitations on an actual size of a product, an actual process of a method and an actual timing of signals that embodiments of the present disclosure relate to.

FIG. 1 is a diagram showing a structure of a positioning head frame, in accordance with some embodiments;

FIG. 2 is a diagram showing a structure of a base frame, in accordance with some embodiments;

FIG. 3A is a diagram showing a structure of another base frame, in accordance with some embodiments;

FIG. 3B is a diagram showing a structure of another base frame, in accordance with some embodiments;

FIG. 4 is a diagram showing a structure of another base frame, in accordance with some embodiments;

FIG. 5A is a diagram showing a structure of another base frame, in accordance with some embodiments;

FIG. 5B is a diagram showing a structure of another base frame, in accordance with some embodiments;

FIG. 6 is a diagram showing a structure of a connector, in accordance with some embodiments;

FIG. 7 is a diagram showing a structure of another connector, in accordance with some embodiments; and

FIG. 8 is a diagram showing a structure of a support member, in accordance with some embodiments.

### DETAILED DESCRIPTION

Technical solutions in some embodiments of the present disclosure will be described clearly and completely in combination with accompanying drawings. Obviously, the described embodiments are merely some but not all embodiments of the present disclosure. All other embodiments obtained obtained on a basis of the embodiments of the present disclosure by a person of ordinary skill in the art shall be included in the protection scope of the present disclosure.

Unless the context requires otherwise, throughout the description and the claims, the term “comprise” and other forms thereof such as the third-person singular form “comprises” and the present participle form “comprising” are construed as an open and inclusive meaning, i.e., “included, but not limited to”. In the description, terms such as “one embodiment”, “some embodiments”, “exemplary embodiments”, “example”, “specific example”, or “some examples” are intended to indicate that specific features, structures, materials or characteristics related to the embodiment(s) or the example(s) are included in at least one embodiment or example of the present disclosure. Schematic representations of the above terms do not necessarily refer to the same embodiment(s) or example(s). In addition, the specific features, structures, materials or characteristics may be included in any one or more embodiments or examples in any suitable manner.

Hereinafter, terms such as “first” and “second” are used for descriptive purposes only and are not to be construed as indicating or implying the relative importance or implicitly indicating the number of indicated technical features below. Thus, features defined as “first” and “second” may explicitly or implicitly include one or more of the features. In the description of the embodiments of the present disclosure, terms “a plurality of”, “the plurality of”, and “multiple” each mean two or more unless otherwise specified.

In the description of some embodiments, terms such as “coupled” and “connected” and their derivatives may be used. For example, the term “connected” may be used in the description of some embodiments to indicate that two or more components are in direct physical or electrical contact with each other. The embodiments disclosed herein are not necessarily limited to the content herein.

In order to ensure that rays emitted by radiotherapy equipment can accurately find tumor tissue to be treated, before precise radiotherapy is performed, parts to be treated of each patient must be fixed and positioned, so that rays can accurately irradiate the targets. As a device for fixing a patient's head during a radiotherapy process, a positioning head frame includes pins and fixed components connected thereto (e.g., the components include a base frame). The pins directly contact the patient's skin to relatively position and fix the patient's head and the positioning head frame.

The base frame is generally all made of a metal material. In a process of nuclear magnetic resonance imaging, the positioning head frame is in an alternating magnetic field. Since the base frame forms a closed loop, the base frame will be affected by the alternating magnetic field and form an electrical loop with the patient's head, thereby generating an induced magnetic field. The induced magnetic field and a magnetic field in a nuclear magnetic resonance process will interact to produce a force and torque. Since the positioning head frame is fixed on the patient's head, the force and the torque will act on the patient's head, thereby causing harm to the patient.

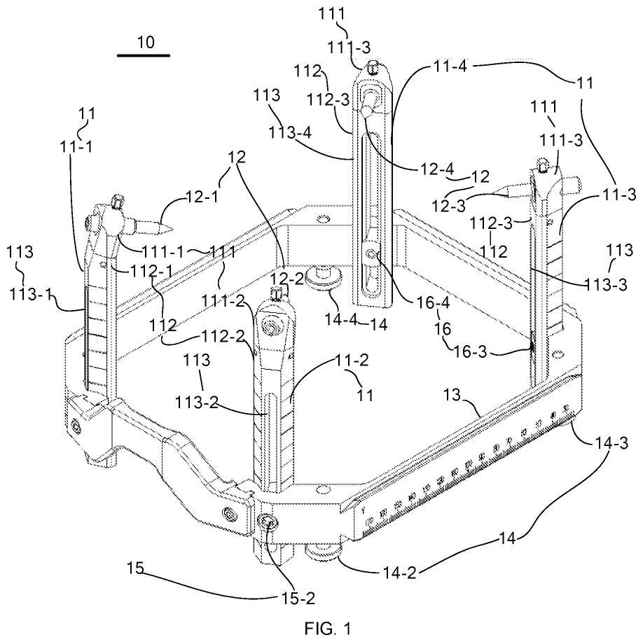

Referring to FIG. 1, some embodiments of the present disclosure provide a positioning head frame 10, which includes a base frame 13, a plurality of support members 11, and a plurality of pins 12. The plurality of support members 11 are fixedly connected to the base frame 13, and each pin 12 is fixedly connected to one end of a corresponding support member 11.

In some examples, the positioning head frame 10 includes four support members 11 and four pins 12. For example, referring to FIG. 1, the plurality of support members 11 include: a support member 11-1, a support member 11-2, a support member 11-3, and a support member 11-4. The plurality of pins 12 include a pin 12-1, a pin 12-2, a pin 12-3, and a pin 12-4. Of course, FIG. 1 is only one example. In the embodiments of the present disclosure, a number of support members 11 and pins 12 may both be two, three, or five, etc. The number of support members 11 and pins 12 may be adaptively changed according to a shape of the base frame.

In some embodiments, referring to FIG. 1, the positioning head frame 10 further includes a plurality of bases 14. A number of bases 14 is not limited in the embodiments of the present disclosure, and may be determined according to actual situations. For example, as shown in FIG. 1, the plurality of bases 14 include a base 14-1 (not shown in FIG. 1), a base 14-2, a base 14-3, and a base 14-4. The base 14 is mainly used as an interface for positioning connection, and is connected to an adapter during magnetic resonance imaging (MRI) or computed tomography (CT) imaging, so that the positioning head frame is placed more stably.

In some embodiments, in order to be more convenient for a doctor to perform treatment, the support member 11 includes a pin fixing part 111 and a base frame fixing part 112. The pin fixing part 111 is configured to be connected to the pin 12, and the base frame fixing part 112 is configured to be fixedly connected to the base frame 13.

For example, as shown in FIG. 1, the support member 11-1 includes a pin fixing part 111-1 and a base frame fixing part 112-1; the support member 11-2 includes a pin fixing part 111-2 and a base frame fixing part 112-2; the support member 11-3 includes a pin fixing part 111-3 and a base frame fixing part 112-3; and the support member 11-4 includes a pin fixing part 111-4 and a base frame fixing part 112-4.

In some examples, an angle between the pin fixing part 111 and the base frame fixing part 112 of at least one support member 11 is 0°. For example, as shown in FIG. 1, the pin fixing part 111-3 and the base frame fixing part 112-3 of the support member 11-3 are both upright, then an angle between the pin fixing part 111-3 and the base frame fixing part 112-3 of the support member 11-3 is 0°.

In some other examples, the angle between the pin fixing part 111 and the base frame fixing part 112 of at least one support member 11 is not 0°. For example, as shown in FIG. 1, the base frame fixing part 112-1 of the support member 11-1 is upright, the pin fixing part 111-1 and the base frame fixing part 112-1 have a certain angle, and then the angle between the pin fixing part 111-1 and the base frame fixing part 112-1 is not 0°.

In some examples, an angle between at least one pin 12 and a pin fixing part 111 of a corresponding support member 11 is 90° or not 90°. For example, a second through hole is disposed in the pin fixing part 111, and the pin 12 passes through the second through hole to connect to the pin fixing part 111, so that an angle between the pin and the pin fixing part is 90° or not 90°.

The pin fixing part 111 and the base frame fixing part 112 of the support member 11 may be integrally formed, or may be formed by connecting two separate parts. In a case where the support member 11 is composed of the pin fixing part 111 and the base frame fixing part 112 that are separate and connected, the pin fixing part 111 is made of an insulating material.

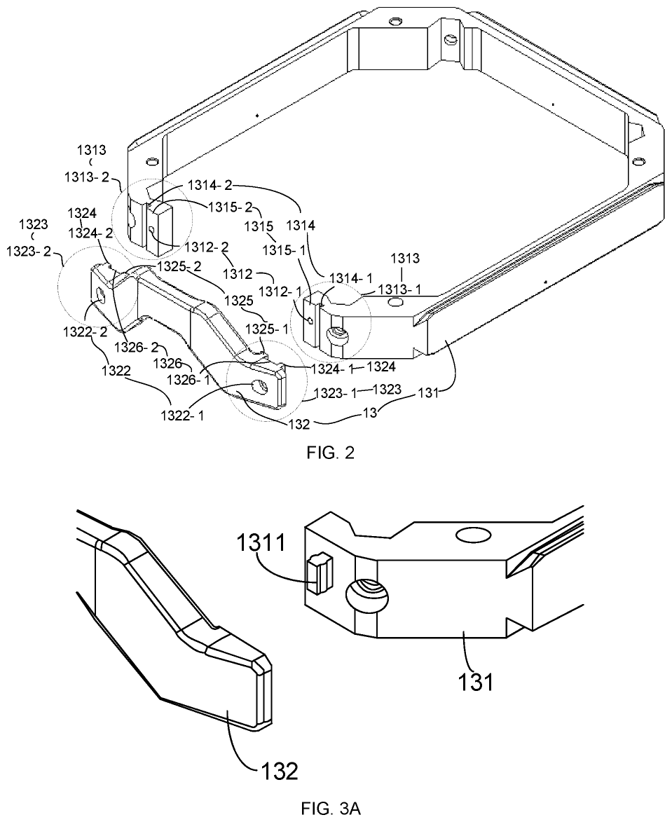

In some embodiments, as shown in FIG. 2, the base frame 13 includes a base frame body 131 and a connector 132. The base frame body has an opening therein, and the base frame body is a conductor. The connector 132 is disposed at the opening of the base frame body 131 to seal the opening of the base frame body 131, and the connector 132 is made of an insulating material.

In some examples, referring to FIG. 2, the base frame 13 is a polygonal ring, and the opening is disposed in one side of the polygonal ring.

For example, the conductor is made of a metal material. For example, the insulating material includes any one or a mixture of the following: ceramic, graphite reinforced fiber plastic, glass reinforced fiber plastic, polyetheretherketone (PEEK) plastic, polyformaldehyde (POM) plastic, and polyetherimide (PEI) plastic. Of course, in the embodiments of the present disclosure, the connector may also be made of a mixture of the above insulating materials and other materials, so long as the insulativity of the connector can be ensured.

Optionally, in order to ensure rigidity of the base frame 13 during use, the base frame body 131 of the base frame 13 is made of a rigid material. In this way, when the base frame body 131 is subjected to an external force, deformation generated may be controlled within a preset range.

The positioning head frame 10 is in a strong magnetic field environment during MRI. In order to prevent various components of the positioning head frame 10 from being affected by the strong magnetic field, and ensure the rigidity of the positioning head frame 10 at the same time to ensure positioning accuracy, for example, the rigid material is a nonmagnetic conductive material, such as a non-ferromagnetic metal material. For example, the non-ferromagnetic metal material includes at least any one of the following: copper, copper alloy, titanium alloy, aluminum alloy, and magnesium alloy.

Since the positioning head frame 10 is installed on the patient's head, in order to reduce a pressure on the patient's head, an overall weight of the positioning head frame needs to be taken into consideration when the non-ferromagnetic metal material is selected. In some examples, a low-density non-ferromagnetic metal material, such as aluminum alloy, may be used. Of course, due to reasons such as structural strength, in some examples, the pins 12 of the positioning head frame 10 and various screws used to connect the components of the positioning head frame 10 are all made of titanium alloy, and metal parts of the base frame 13 and the support members 11 are mostly made of aluminum alloy.

In some embodiments, two ends of the connector 132 are detachably connected to two ends, at the opening, of the base frame body 131 respectively.

In some examples, one end of the connector 132 and one end, at the opening, of the base frame body 131 are detachably connected through a snap fit structure.

The snap fit structure includes an elastic protrusion disposed on the connector 132 and a locking slot disposed in the base frame body 131; or, the snap fit structure includes an elastic protrusion disposed on the base frame body 131 and a locking slot disposed in the connector 132. The elastic protrusion is configured to be matched with the locking slot in a snap fit mode to prevent the base frame body 131 and the connector 132 from moving relative to each other.

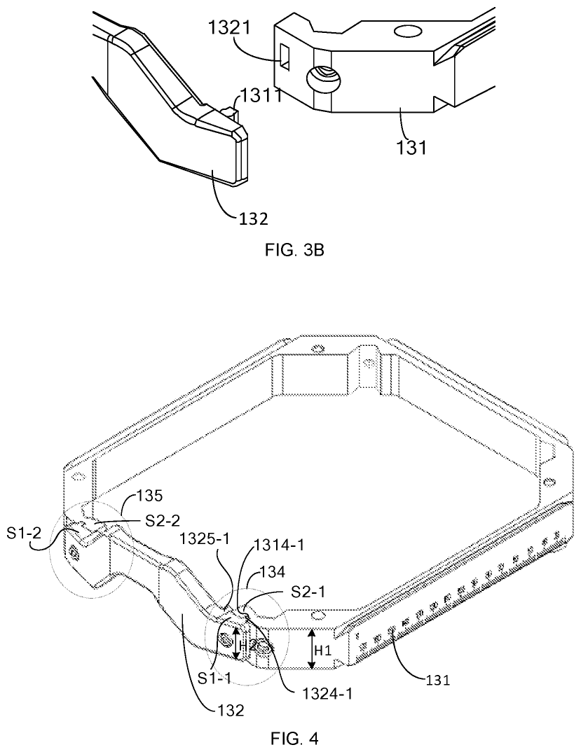

For example, referring to FIG. 3A, an elastic protrusion 1311 is disposed on one end of the base frame body 131, and a locking slot 1321 (not shown) is disposed in one end of the connector 132. For another example, referring to FIG. 3B, the locking slot 1312 is disposed in one end of the base frame body 131, and the elastic protrusion 1311 is disposed on one end of the connector 132.

Referring to FIG. 4, the base frame body 131 and the connector 132 have two connected parts 134 and 135. The two connected parts may be connected in a connection manner shown in FIG. 3A, or may be connected in a connection manner shown in FIG. 3B, or may be connected in connection manners shown in FIGS. 3A and 3B respectively.

In some other examples, one end of the connector 132 and one end, at the opening, of the base frame body 131 are detachably connected through a thread structure.

The thread structure includes a bolt, a first through hole disposed in the connector 132, and a first threaded hole disposed in the base frame body 131, and the bolt is configured to pass through the first through hole and be threadedly matched with the first threaded hole. Or, the thread structure includes a bolt, a first threaded hole disposed in the connector 132, and a first through hole disposed in the base frame body 131, and the bolt is configured to pass through the first through hole and be threadedly matched with the first threaded hole.

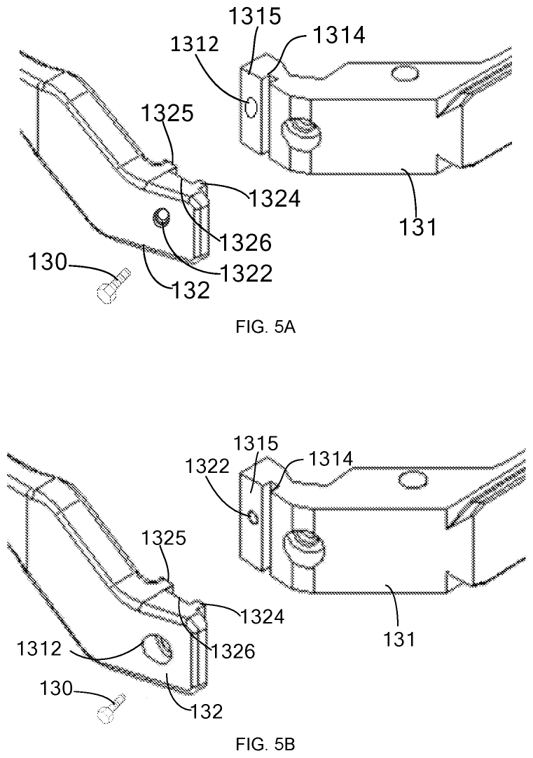

For example, referring to FIG. 5A, a first through hole 1312 is disposed in one end of the base frame body 131, and a first threaded hole 1322 is disposed in one end of the connector 132. For another example, referring to FIG. 5B, the first threaded hole 1322 is disposed in one end of the base frame body 131, and the first through hole 1312 is disposed in one end of the connector 132.

In some examples, heights H2 of the two ends of the connector 132 are different from heights H1 of the two ends, at the opening, of the base frame body 131.

In some other examples, as shown in FIG. 4, the heights H2 of the two ends of the connector 132 are the same as the heights H1 of the two ends, at the opening, of the base frame body 131. FIG. 4 only shows that a height of one end of the connector 132 is H2, and a height of one end, at the opening, of the base frame body 131 is H1. It can be understood that a height of the other end of the connector 132 is also H2, and a height of the other end, at the opening, of the base frame body 131 is also H1.

In a case where H1 and H2 are the same, when the bolt 130 passes through the first through hole 1312 and is threadedly matched with the first threaded hole 1322 to cause the connector 132 and the base frame body 131 to be fixedly connected, an upper surface of a portion connected by the connector 132 and the base frame body 131 is flush, and a lower surface of a portion connected by the connector 132 and the base frame body 131 is also flush. In this way, the base frame 13 may be made more beautiful and easy to install. For example, referring to FIG. 4, when the connector 132 and the base frame body 131 are connected, upper surfaces S1 (S1-1 and S1-2) at the two ends of the connector 132 are flush with upper surfaces S2 (S2-1 and S2-2) at the two ends, at the openings, of the base frame body 131, and lower surfaces S3 (S3-1 and S3-2, not shown in FIG. 4) at the two ends of the connector 132 are flush with lower surfaces S4 (S4-1 and S4-2, not shown in FIG. 4) at the two ends, at the opening, of the base frame body 131.

Referring to FIG. 4, the base frame body 131 and the connector 132 have two connected parts 134 and 135. The two connected parts may be connected in a connection manner shown in FIG. 5A, or may be connected in a connection manner shown in FIG. 5B, or are connected in connection manners shown in FIGS. 5A and 5B respectively. Taking the two connected parts being connected in the connection manner shown in FIG. 5B as an example, the structures of the base frame body 131 and the connector 132 are as shown in FIG. 2.

In a case where one end of the connector 132 is detachably connected to one end of the base frame body 131 through the thread structure, in some examples, the base frame 13 further includes a pre-positioning structure for pre-positioning the connector 132 and the base frame body 131 in a snap fit mode. When there is a need to connect the connector 132 and the base frame body 131, the pre-positioning structure is used to pre-position the two first, and then bolts are used to be matched with the first through hole 1312 and the first threaded hole 1322 to connect the two.

Since the base frame 13 includes the base frame body 131 and the connector 132, the support members 11 are disposed on the base frame body 131, and the pins are disposed on the support members 11 and are in contact with the patient's head. When the pins are fixed on the head, the support members 11 will subject to forces from the pins, and the forces will be transmitted to the base frame body 131 through the support members 11, so that the base frame body 131 may be deformed to a certain extent. By providing the pre-positioning structure, it may fix a relative relationship between the connector 132 and the base frame body 131 to a certain extent. Therefore, the base frame body 131 will not be deformed due to the forces transmitted by the support members 11, thereby improving an anti-deformation ability of the base frame 13. In addition, it is convenient for the bolt to pass through the first through hole 1312 and be threadedly matched with the first threaded hole 1322, so that the connector 132 and the base frame body 131 may be better connected.

In some examples, the pre-positioning structure includes a first positioning groove disposed in one of the connector 132 and the base frame body 131, and a first positioning rib disposed on the other of the connector 132 and the base frame body 131. The first positioning rib is configured to be matched with the first positioning groove, so that a central axis of the first through hole 1312 and a central axis of the first threaded hole 1322 coincide with each other. That is, when the first positioning groove and the first positioning rib are matched with each other, the central axis of the first through hole 1312 and the central axis of the first threaded hole 1322 coincide with each other.

It will be noted that, in order to facilitate disassembly, the first positioning rib and the first positioning groove have a micro gap therebetween when being installed in cooperation with each other.

Referring to FIG. 2, taking an example of that a first positioning rib 1324 is disposed on the connector 132, a first positioning groove 1314 is disposed in the base frame body 131, the first through hole 1312 is disposed in the base frame body 131, and the first threaded hole 1322 is disposed in the connector 132, two ends 1313-1 and 1313-2, at the opening, of the base frame body 131 are respectively provided with a first positioning groove 1314-1 and a first positioning groove 1314-2, and two ends 1323-1 and 1323-2 of the connector 132 are respectively provided with a first positioning rib 1324-1 and a first positioning rib 1324-2. When the first positioning groove 1314-1 and the first positioning rib 1324-1, and the first positioning groove 1314-2 and the first positioning rib 1324-2 are respectively matched with each other, the central axis of the first through hole 1312 and the central axis of the first threaded hole 1322 coincide with each other.

In some examples, in order to ensure the rigidity of the base frame body 131 or the connector 132, the pre-positioning structure further includes a second positioning rib. The second positioning rib is disposed on a same one of the connector 132 and the base frame body 131 as the first positioning rib 1324, and the first positioning rib 1324 is configured to be matched with the first positioning groove 1314. After the first positioning rib 1324 and the first positioning groove 1314 are matched with each other, a side face of the second positioning rib facing the first positioning rib is opposite to an end face of an end of the connector 132 where the first positioning groove 1314 is located or an end at the opening of the base frame body 131. Further, in order to facilitate disassembly, the side face of the second positioning rib facing the first positioning rib and the end face of the end of the connector where the first positioning groove is located or the end, at the opening, of the base frame body have a gap therebetween.

Referring to FIG. 2, taking an example of that the first positioning rib 1324 is disposed on the connector 132, the first positioning groove 1314 is disposed in the base frame body 131, the first through hole 1312 is disposed in the base frame body 131, and the first threaded hole 1322 is disposed in the connector 132, the two ends 1323-1 and 1323-2 of the connector 132 are respectively provided with a second positioning rib 1325-1 and a second positioning rib 1325-2. Referring to FIG. 4, after the first positioning rib 1324-1 and the first positioning groove 1314-1 are matched with each other, a side face of the second positioning rib 1325-1 facing the first positioning rib 1324-1 is opposite to and has a gap with an end face of an end 134, at the opening, of the base frame body 131 where the first positioning groove 1314-1 is located.

Referring to FIG. 2, it is equivalent that a second positioning groove 1326 (1326-1 and 1326-2) is formed between the first positioning rib 1324 and the second positioning rib 1325, and a third positioning rib 1315 (1315-1 and 1315-2) is formed between the first positioning groove 1314 and an end face of an end 1313, at the opening, of the base frame body 131. After the first positioning rib 1324 is cooperatively installed into the first positioning groove 1314, the third positioning rib 1315 is cooperatively installed into the second positioning groove 1326.

For example, referring to FIG. 5A, the first through hole 1312 is disposed in the third positioning rib 1315, and the first threaded hole 1322 is disposed in the second positioning groove 1326. For another example, referring to FIG. 5B, the first through hole 1312 is disposed in the second positioning groove 1326, and the first threaded hole 1322 is disposed in the third positioning rib 1315.

In order to ensure beautiful surface and convenient use of the base frame 13, optionally, the first through hole 1312 is counterbore, i.e., a stepped bore. A radius of a larger hole in the counterbore is slightly larger than a radius of a nut of the bolt, and a length of the larger hole in the counterbore is equal to or slightly greater than a height of the nut. A radius of a smaller hole in the counterbore is larger than a maximum radius of a screw of the bolt. The first threaded hole 1322 is a blind hole or a through hole.

A connection manner between the base frame body 131 and the connector 132 is not limited to the above two implementation manners, and may also be other manners. For example, the two ends where the base frame body 131 and the connector 132 are connected are respectively provided with a protrusion and a groove, and the protrusion and the groove make the base frame body 131 and the connector 132 to be fixedly connected in an interference fit manner.

In order to adapt to different head types and different diseased parts of patients, the connector 132 of the positioning head frame 10 may have a V-shaped structure or a flat type structure.

For example, in a case where the positioning head frame 10 is disposed on the patient's head and the connector 132 faces the patient's mouth, the connector 132 has the flat type structure. For another example, in a case where the positioning head frame 10 is disposed on the patient's head, and the connector 132 faces the patient's nose, the connector 132 has the V-shaped structure. In this way, the connector 132 can avoid the nose, so as to avoid severe pressure on the patient's nose.

For example, referring to FIG. 4, in a case where the connector 132 has the flat type structure, H1 and H2 are the same. For another example, in a case where the connector has a special-shaped structure such as the V-shaped structure, H1 and H2 are different.

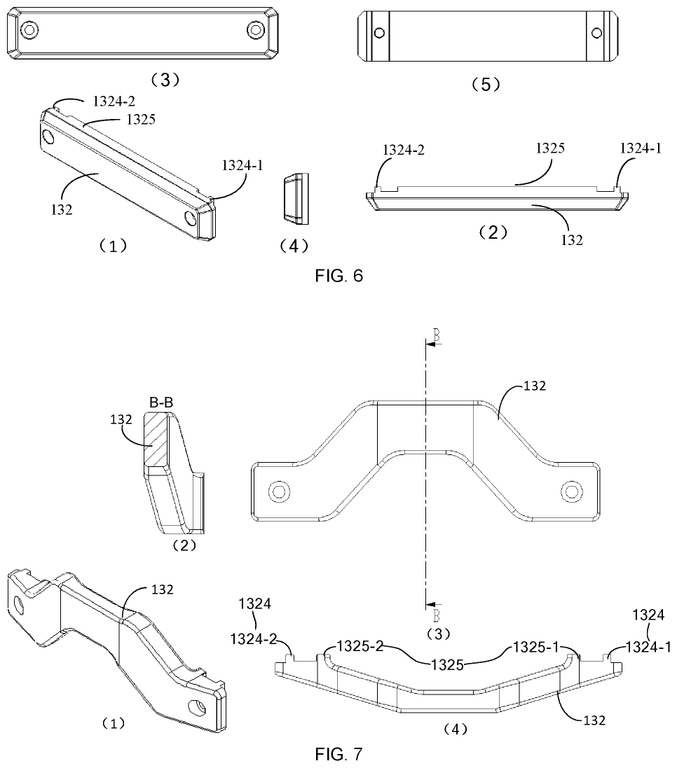

Taking the first positioning rib 1324 and the second positioning rib 1325 being disposed on the connector 132 as an example, a specific structure of the connector 132 with the flat type structure is shown in FIG. 6 (FIGS. 6(1), 6(2), and 6(3), 6(4) and 6(5)). FIG. 6(1) is a schematic stereoscopic diagram of the connector 132 with the flat type structure, FIG. 6(2) is a top view of the connector 132 with the flat type structure, FIG. 6(3) is a front view of the connector 132 with the flat type structure, FIG. 6(4) is a side view of the connector 132 with the flat type structure, and FIG. 6(5) is a front view of the connector 132 with the flat type structure from another direction.

Taking the first positioning rib 1324 and the second positioning rib 1325 being disposed on the connector 132 as an example, a specific structure of the connector 132 with the V-shaped structure is shown in FIG. 7 (FIGS. 7(1), 7(2), 7(3), and 7(4)). FIG. 7(1) is a schematic stereoscopic diagram of the connector 132 with the V-shaped structure, FIG. 7(2) is a cross-sectional view of the connector 132 with the V-shaped structure, FIG. 7(3) is a front view of the connector 132 with the V-shaped structure, and FIG. 7(4) is a top view of the connector with the V-shaped structure. For example, as shown in FIG. 7(3), a middle part of the connector 132 with the V-shaped structure is V-shaped. As shown in FIG. 7(4), the whole connector 132 with the V-shaped structure protrudes towards a side away from the first positioning rib 1324 (1324-1 and 1324-2) and the second positioning rib 1325 (1325-1 and 1325-2). Referring to FIG. 1, the connector 132 with the V-shaped structure may avoid the patient's discomfort caused by the patient's nose being restricted by the positioning head frame 10.

In some embodiments, in order to allow the support member 11 to move on the base frame 13 to a certain extent, referring to FIG. 1, a kidney-shaped hole 113 (113-1, 113-2, 113-3, and 113-4) is disposed in the support member 11, and may be matched with an adjusting bolt 15 (15-1, 15-2, 15-3, and 15-4, only 15-2 is shown in FIG. 1) and an adjusting block 16 (16-1, 16-2, 16-3, and 16-4, only 16-3 and 16-4 are shown in FIG. 1) to adjust the relative positions of the support member 11 and the base frame 13.

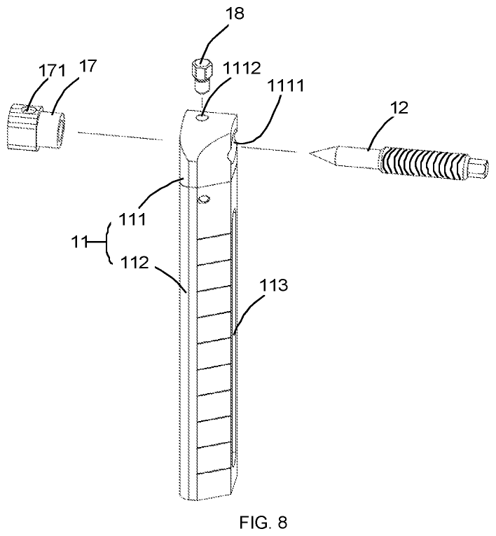

Referring to FIG. 8, the embodiments of the present disclosure provide a support member 11 including a pin fixing part 111 and a base frame fixing part 112. The pin fixing part 111 is fixedly connected to the pin 12 through a sleeve 17, and the base frame fixing part 112 is fixedly connected to the base frame 13. The pin fixing part 111 is made of the insulating material mentioned in the aforementioned embodiments, and the sleeve 17 is made of the aforementioned rigid material.

In this way, on one hand, an electrical connection cannot be formed between the pin 12 and the support member 11. When the positioning head frame 10 is in a strong magnetic field, and an electrical loop is formed between the positioning head frame 10 and the patient's head to generate current and heat, the patient will not get burnt; on another hand, since the sleeve 17 is made of the rigid material, it may be used repeatedly to avoid that the pin 12 is not firmly fixed due to the wear of the sleeve 17.

In order to ensure the rigidity of the support member 11, in some examples, the base frame fixing part 112 of the support member 11 is also made of the aforementioned rigid material.

In an example, the pin fixing part 111 and the base frame fixing part 112 are connected in an interference fit manner. For example, the two ends where the pin fixing part 111 and the base frame fixing part 112 are connected are respectively provided with a protrusion and a groove, and the protrusion and the groove make the pin fixing part 111 and the base frame fixing part 112 to be fixedly connected in an interference fit manner. Of course, any other connection manners that can achieve the purpose of fixed connection between the pin fixing part 111 and the base frame fixing part 112 may also be adopted, and there is no specific limitation herein.

In some examples, a second through hole is disposed in the pin fixing part, and the second through hole is provided with a sleeve. The pin is configured to pass through the sleeve and be connected to the pin fixing part. For example, referring to FIG. 8, the second through hole 1111 is disposed in the pin fixing part 111, and the sleeve 17 is cooperatively matched into the second through hole 1111. After the sleeve 17 is installed into the second through hole 1111, the pin 12 may pass through a threaded hole in the sleeve 17 to achieve the purpose of fixed installation.

In some examples, as shown in FIG. 8, a first blind hole 1112 is further disposed in the pin fixing part 111, and the first blind hole 1112 and the second through hole 1111 intersect and communicate with each other. A second blind hole 171 is disposed in the sleeve 17, and the second blind hole 171 is configured to communicate with the first blind hole 1112 after the sleeve is installed into the second through hole 1111. For example, referring to FIG. 8, the first blind hole 1112 extends from a top face of the pin fixing part 111 away from the base frame fixing part 112 to the inside of the pin fixing part 111, and intersects and communicates with the second through hole 1111. For another example, the first blind hole 1112 extends from a side face of the pin fixing part 111 to the inside of the pin fixing part 111, and intersects and communicates with the second through hole 1111. It will be noted that, the side face of the pin fixing part 111 is a side face excluding a top face of the pin fixing part 111, a bottom face opposite to the top face, and two opposite side faces provided with the second through hole.

Before the pin 12 is installed, the sleeve 17 is installed into the second through hole 1111, a fastening pin 18 is installed into the first blind hole 1112, and a part of the fastening pin 18 enters the second blind hole 171. In this case, the fastening pin 18 may be in contact with the sleeve, so as to ensure that the sleeve 17 cannot fall off from the second through hole 1111. After the pin 12 is installed into the sleeve 17, the fastening pin 18 may also prevent the sleeve 17 from moving at will when the position of the pin 12 is adjusted.

In some examples, in order to prevent the sleeve 17 from rotating with the adjustment of the position of the pin 12, the second through hole may be a circular hole, or irregular hole such as non-regular polygonal holes, and an outer contour of at least one cross section of the sleeve 17 along a direction perpendicular to its central axis is non-circular, such as ellipse, square, rectangle, pentagon, hexagon, isosceles trapezoid, and right-angle trapezoid.

For example, the second through hole 1111 is a circular hole, and the sleeve 17 is non-circular. In this case, the sleeve 17 is not easy to rotate in the second through hole 1111. For another example, the second through hole 1111 is an irregular hole, and the sleeve 17 is non-circular. In this case, the sleeve 17 is not rotatable in the second through hole 1111.

In some examples, after the sleeve 17 is installed into the second through hole 1111 and the pin 12 is installed into the sleeve 17, the central axes of the second through hole 1111, the sleeve 17 and the pin 12 are coaxial.

In the base frame 13 and the positioning head frame 10 provided by the embodiments of the present disclosure, the base frame 13 includes the base frame body 131 and the connector 132. The base frame body 131 has an opening therein, and the base frame body 131 is the conductor. The connector is disposed at the opening of the base frame body 131, so as to seal the opening of the base frame body 131, and the connector 132 is made of the insulating material. In the technical solutions provided by the embodiments of the present disclosure, since one of two parts, i.e., the connector 132, constituting the base frame 13 is made of the insulating material, the base frame 13 cannot form an electrical loop. In this way, when the positioning head frame 10 is in the alternating magnetic field, the base frame 13 will not generate current due to the impact of the alternating magnetic field and will not generate the induced magnetic field. As a result, it may prevent an additional force generated by interaction between the induced magnetic field generated by the ring base 13 and the magnetic field of the nuclear magnetic resonance equipment from acting on the patient's head and causing injury to the patient.

The foregoing descriptions are merely some specific implementations of the present disclosure, but the protection scope of the present disclosure is not limited thereto. Any changes or replacements that a person skilled in the art could conceive of within the technical scope of the present disclosure shall be included in the protection scope of the present disclosure. Therefore, the protection scope of the present disclosure shall be subject to the protection scope of the claims.

What is claimed is:

1. A base frame, comprising:
a base frame body having an opening therein, the base frame body being a conductor; and
a connector disposed at the opening of the base frame body to seal the opening of the base frame body; the connector being made of an insulating material.

2. The base frame according to claim 1, wherein two ends of the connector are detachably connected to two ends, at the opening, of the base frame body respectively.

3. The base frame according to claim 2, wherein one end of the connector and one end, at the opening, of the base frame body are detachably connected through a thread structure.

4. The base frame according to claim 3, wherein the thread structure includes a bolt, a first through hole, and a first threaded hole;
the first through hole is disposed in the connector, and the first threaded hole is disposed in the base frame body; or, the first threaded hole is disposed in the connector, and the first through hole is disposed in the base frame body; and
the bolt is configured to pass through the first through hole and be threadedly matched with the first threaded hole.

5. The base frame according to claim 4, further comprising:
a pre-positioning structure for pre-positioning the connector and the base frame body in a snap fit mode.

6. The base frame according to claim 5, wherein the pre-positioning structure includes a first positioning groove and a first positioning rib; the positioning groove is disposed in one of the connector and the base frame body, and the first positioning rib is disposed on another of the connector and the base frame body; and
the first positioning rib is configured to be matched with the first positioning groove to make a central axis of the first through hole and a central axis of the first threaded hole coincide with each other.

7. The base frame according to claim 6, wherein the pre-positioning structure further includes a second positioning rib; and the second positioning rib is disposed on a same one of the connector and the base frame as the first positioning rib; and
after the first positioning rib and the first positioning groove are matched with each other, a side face of the second positioning rib facing the first positioning rib is opposite to and has a gap with an end face of an end of the connector where the first positioning groove is located or an end, at the opening, of the base frame body.

8. The base frame according to claim 2, wherein one end of the connector and one end, at the opening, of the base frame body are detachably connected through a snap fit structure.

9. The base frame according to claim 8, wherein the snap fit structure includes an elastic protrusion and a locking slot;
the elastic protrusion is disposed on the connector, and the locking slot is disposed in the base frame body; or, the elastic protrusion is disposed on the base frame body, and the locking slot is disposed in the connector; and
the elastic protrusion is configured to be matched with the locking slot in a snap fit mode to prevent the base frame body and the connector from moving relative to each other.

10. A positioning head frame, comprising:
the base frame according to claim 1;
a plurality of support members fixedly connected to the base frame; and
a plurality of pins, each pin being fixedly connected to one end of a corresponding support member.

11. The positioning head frame according to claim 10, wherein one of the plurality of support members includes a pin fixing part and a base frame fixing part; and
the pin fixing part is configured to be connected to the pin, and the base frame fixing part is configured to be fixedly connected to the base frame.

12. The positioning head frame according to claim 11, wherein an angle between the pin fixing part and the base frame fixing part of at least one support member is 0°.

13. The positioning head frame according to claim 11, wherein an angle between the pin fixing part and the base frame fixing part of at least one support member is not 0°.

14. The positioning head frame according to claim 11, wherein an angle between at least one pin and a pin fixing part of a corresponding support member is 90°.

15. The positioning head frame according to claim 11, wherein an angle between at least one pin and a pin fixing part of a corresponding support member is not 90°.
